(19) United States
(12) Patent Application Publication
(10) Pub. No.: US 2011/0177179 A1
(43) Pub. Date: Jul. 21, 2011

(54) PREPARATION OF BOTANICAL EXTRACTS CONTAINING ABSORBABLE COMPONENTS USING PHARMACEUTICAL PLATFORM TECHNOLOGY

(75) Inventors: Yun Kau TAM, Hong Kong (CN); Yi-Chan James LIN, Edmonton (CA); Brian Duff SLOLEY, Edmonton (CA)

(73) Assignee: SINOVEDA CANADA, INC., Edmonton (CA)

(21) Appl. No.: 13/008,737

(22) Filed: Jan. 18, 2011

Related U.S. Application Data
(60) Provisional application No. 61/295,940, filed on Jan. 18, 2010.

(51) Int. Cl. A61K 36/48; A61P 35/00; A61P 9/12; A61P 25/06; G01N 33/53

(52) U.S. Cl. 424/757; 435/7.2; 435/7.21

(57) ABSTRACT

Processes describing approaches to prepare herbal extracts containing absorbables and/or precursors of absorbables are described. The procedures are based on the physiological events in the gastrointestinal tract. Improvement of component absorption can also be achieved by designing an appropriate extraction condition.

This application claims benefit of U.S. provisional patent application No. 61/295,940, filed on Jan. 18, 2010, the contents of which are incorporated herein by reference in its entirety.

Throughout this application, various references are referred to and disclosure of these publications in their entireties are hereby incorporated by reference into this application to more fully describe the state of the art to which this invention pertains.

### FIELD OF THE INVENTION

The present invention relates to the development, preparation, and refining of a Platform Pharmaceutical Technology for the Development of Natural Products (Tam and Tuszynski 2008).

### BACKGROUND OF THE INVENTION

Natural products have been used by human civilization for thousands of years. Their medicinal values have been recorded throughout history. Through advancement of pharmacology, clinical pharmacology, pharmacognosy and analytical chemistry, the active components in natural substances have begun to be unveiled. A good example is the discovery of salicylic acid in willow bark. Bayer has recently celebrated the 100^{th} anniversary of Aspirin, a purified form of acetylated salicylic acid.

There are two streams of natural product research. Since the dawn of modern pharmaceutical sciences, there has been an insatiable quest for the isolation and purification of a single active component in a natural substance. In fact, more than 60% of the pharmaceuticals which have been developed for treating cancer, hypertension and migraines are either natural in origin or natural product mimics (Newman, Cragg et al. 2003). Although combinatorial techniques have succeeded as methods of optimizing structures, no de novo combinatorial compound approved as a drug has been identified on or before 2002. In the hope of finding new core chemical structures, efforts are still being made in natural product research. Unigen has developed a high throughput platform technology, Pharmacologix, to identify leads from a natural product (Jia and Hong 2003). This technology involves a two solvent extraction process, an in vitro pharmacological assay and a dereplication technique. The first extraction process consists of the use of a solvent mixture of polar and nonpolar solvents.

Natural remedies are often composed of one or more herbs. Each herb has multiple active components. The identification, purification, and activity determination, using known pharmacological models for a complex mixture, has been a monumental task. The complexity of this area of research has been the major obstacle in natural medicine development (Williamson 2001). In their review, Liu and Yang (2006) commented that identifying active components in traditional Chinese Medicine (TCM) is the most important issue in the development of TCM. The active components could be active metabolites of the principal components of the preparation. For example, ginsenosides are major components responsible for the efficacy of ginseng. However, the activity of these ginsenosides is low and their bioavailability after oral administration is minuscule. The metabolic products, protopanaxadiols and protopanaxatriols are easily absorbed and pharmacologically active (Hasegawa 2004). Although it is important to understand the pharmacokinetic and pharmacodynamic nature of the active components in TCM, thus far, the art shows no capability for sorting out potential pharmacokinetic and pharmacodynamic interactions, which are extremely complicated. As described in this invention, inactive constituents could have a significant effect on the absorption of active components.

The study of active ingredients in natural substances has been rather primitive in pharmaceutical science terms. The approach has stagnated at the discovery stage of pharmaceutical development. The general approach is to employ activity guided extraction to identify targets that have in vitro activities. This approach is extremely unsuitable for the development of natural products. For the longest time, Panax ginseng was thought to be an expensive “junk” because it has no apparent active ingredients. It was not until Hasegawa (2004) reported that the inactive ginsenosides of Panax ginseng were acting like prodrugs, was it revealed that when metabolized by intestinal flora aglycones were released, which have physiological activity. Rutin, a flavonoid glycoside, which is present in ginkgo and a number of other herbs, has been shown to be a potent antioxidant in vitro. However, it is difficult to substantiate the actual in vivo activity of rutin, simply because this substance is not detected in the blood stream (Hollman, van Trijp et al. 1997). A major component of Chuanxiong, z-ligustilide, has been shown to be a major active component of the herb; however, the bioavailability of this component is less than 3% (Yan, Ko et al. 2008). It is quite obvious that there will not be enough z-ligustilide reaching the site of action to exert its activity. These examples clearly show the shortcoming of using the classical pharmaceutical approach of identifying actives in an herbal preparation. Natural prodrugs, like the ginsenosides, will be missed and actives like rutin will be pursued. In pharmaceutical science terms, compounds like z-ligustilide lack drug-like properties for oral administration. Drug-like properties are basically pharmacokinetic properties of a substance, which indicate whether, after administration, the substance has the ability to be absorbed in a substantial amount without being metabolized, and to be distributed via the blood stream to the site of action in sufficient quantity before being eliminated from the body. It is no surprise that drug-like properties have not been a major component of natural product research because it is new to pharmaceutical development. Since there are permutations in arriving at the actives of an herbal extract, the complexity of delineating pharmacokinetic profiles for multi-components does appear to be prohibitive.

Recognizing the complex nature of herbal product development, Homma et al. (1992) proposed a strategy to identify biologically active components in an herbal product. The premise of the strategy is that ingredients and/or their metabolites have to be absorbed before they can exert their biological effects. The contents of ingredients and/or their metabolites in plasma and urine after product administration were measured. This approach has been employed by Pan and Cheng (2006) to evaluate a Chinese herbal product, Shuangdan. It was proposed that some of the components that were present in plasma could be used for standardization of the product. This approach can certainly be used to identify absorbable components and their metabolites. Zhang et al. (2005) examined using chemical and metabolic fingerprinting for identifying potentially active ingredients in Danshen injection batches.

The advance of analytical technology complicates the plasma approach. Although the number of absorbable components may be drastically reduced, the number of metabolites that are produced from the body could be daunting. One could argue that only the major components need be standardized; however, this assumption is clearly flawed because potent components present in minute quantities may be missed. Among other shortcomings, this approach for identifying biologically active components does not permit optimization of ratio and dosage of biologically active components. Furthermore, it is difficult to distinguish between metabolites that are produced in the gut as opposed to a metabolizing organ such as the liver. Hence, it is difficult to account for absorbable metabolites.

In recent years, interests in performing pharmacological and pharmacokinetic studies on natural substances such as St. John's Wort (Schulz, Schurer et al. 2005) and Ginkgo (Kwak, Han et al. 2002; Ahlemeyer and Krieglstein 2003) are increasing. There is no lack of publications in the area of herb-drug interactions (Brazier and Levine 2003; Hu, Yang et al. 2005; Williamson 2005), herbal effects on drug metabolizing enzymes (Venkataramanan, Ramachandran et al. 2000; Mathews, Etheridge et al. 2002; Komoroski, Zhang et al. 2004; Yim, Kim et al. 2004; Chang, Chen et al. 2006) and pharmacokinetics of active ingredients of herbs (Mathews, Etheridge et al. 2005; Zhou, Huang et al. 2005; Yan, Lin et al. 2007). Publications on the latter subject are limited to studies involving the pharmacokinetics of a single component. There are studies, which attempted to predict in vivo herb-drug interaction using in vitro methodologies (Williamson 2001; Mohutsky, Anderson et al. 2006; Venkataramanan, Komoroski et al. 2006). These studies met with partial success and the general conclusion is that an in vivo study is required to confirm the results.

It has been frequently postulated that the advantage of alternative therapy is the relatively low dosage required for the treatment of an ailment (Williamson 2001). Active components could act either additively, synergistically or antagonistically. This subject remains elusive to scientists working on the development of herbal medicine. Wang et al. (2006) have designed a method called Quantitative Composition-activity Relationship (QCAR) to identify herbs that are active in a multiple herb formula. While individual herbs contain mixtures of compounds, there was no attempt to address the effects of potential variability within each herb on the pharmacological outcome of the formula. Although in vivo interaction between herbs was reported, there were no indications as to which components in each herb were involved. The same group of scientists has also published a method to address the issue faced with mixtures in QCAR (Cheng, Wang et al. 2006). However, the active components identified using these methodologies were restricted to activity only; there was no attempt to investigate the “drug-like” properties of active components. Since a large number of herbs contain ingredients that behave like precursors, e.g., ginsenosides from Panax ginseng in their native forms, they are inactive. This method would have missed this category of “active” ingredients. In the absence of an understanding of the number of components/precursors involved and their respective drug-like properties, it would be close to impossible to determine these intricate interactions in the body. The methods developed by this group of scientists were based on linear models. This limitation has restricted the evaluation of interactions, including synergism and antagonism. Furthermore, they do not take the nonlinear relationship between intensity of activity and concentration into account, a relationship that is important for understanding optimal dosing and degree of component-component interaction (Chou 2006).

Pharmaceutical technologies for drug discovery have not been employed extensively in the development of natural products. There are a number of in vitro microsomal or hepatocyte studies reported for evaluating herb-drug interactions (Hu, Yang et al. 2005; Williamson 2005; Venkataramanan, Komoroski et al. 2006) and metabolism of active components (Komoroski, Parise et al. 2005). However, there is no study on using physiologically based pharmacokinetic and/or pharmacodynamic models to predict the time course of active ingredients of an herbal extract in the body, nor are there any studies using the same approach to quantify the time course of a response. No in silico methods to-date employed for drug discovery have been applied to predict the pharmacokinetic and pharmacodynamic interaction of active components and their metabolites after administration of an herbal extract.

There have been a number of patents filed in the last 20 years outlining methods for standardizing natural products. The most advanced ones are that of Paracelsian's BioFit® (Blumenthal and Milot 2004), CV Technologies' ChemBioPrint® (Pang, Shan et al. 2000) and PharmaPrint Inc's. PharmaPrint® technologies (Khwaja and Friedman 2000; Khwaja and Friedman 2002). The later two utilize bioassays involving concentrating fractions that are pharmacologically active and standardizing one or more markers along with desired activities. When both conditions are satisfied, the batch is accepted. PharmaPrint® rates these extracts pharmaceutical grade. They have used this technology to produce standardized herbs such as St. John's Wort (Khwaja and Friedman 2000). ChemBioPrint® appears to be a bit more involved (vis-à-vis BioFit® and PharmaPrint®) in that in addition to the in vitro assays, in vivo assays are also incorporated in the standardization procedures. Neither of these two standardization procedures directly links the activity with the putative standardized ingredients. Therefore, it is not known whether the standardized ingredients are of the right amount or the appropriate ratios. There is also no information on active ingredients that are not identified. It is well known that some of these ingredients are inactive in vitro, but they have biological activities in vivo (Hasegawa 2004). The reason is that some of these ingredients are not actually absorbed; therefore lacking “drug-like” properties. Paracelsian's BioFit® technology claimed that an absorption assessment using Caco-2 cells was performed on the active components. However, Caco-2 has shortcomings in predicting absorption of relatively large molecules because these molecules are not permeable through the Caco-2 membrane. A significant percentage of natural ingredients have relatively large molecular weights. The absorption of these molecules, such as polysaccharides, glycosides, etc. is difficult to estimate using Caco-2 cells.

Kinetana's SimBioDAS® technology (Tam and Anderson 2000) appears to overcome the problems that Caco-2 technology faces (Blumenthal and Milot 2004). This technology has been employed to measure absorbable components, which are active in vitro. This technology, however, has two problems: 1. it does not provide an estimate of the pharmacokinetics of ingredients and therefore, concentration-time profiles at the site of action; and 2. the cell membranes are susceptible to rupture when they are incubated with certain herbal extracts such as St. John's Wort.

There was a news release in January 2008 by an Indian firm named Avesthagen announcing a new technology, MetaGrid, for the standardization of multi-constituent plant-based extracts. This technology is based on matching retention times of active components analyzed using an analytical method. While the technology may be useful for standardizing active components, however, the so-called active components have not been subjected to vigorous testing for in vivo testing. In other words this technology does not provide information on the “drug-like” properties of these components.

In short, there is no method available to adequately mine the physiologically active components of an herbal substance. It is generally believed that the activity of phytomedicine is mediated by a large number of active ingredients, each of which constitutes a relatively low quantity compared to those used in Western medicines. Furthermore, each ingredient, if given individually, would require a much higher dose to achieve the same physiological effect. It is believed, however, (while rarely demonstrated directly by experiment) that these individual ingredients, when taken together, may mutually reinforce each other synergistically. For example, in a given herbal extract (e.g. Echinacea or Ginkgo biloba), there could be several hundred chemical entities, dozens of which are active compounds and a subset of these can strongly interact with each other synergistically or by mutual inhibition. However, existing technology does not allow stringent quality control because there have been no success in elucidating the activity of these ingredients as a group. Tam et al. (Tam and Tuszynski 2008) have invented a platform technology, which is based on formulating a mathematically rigorous procedure of describing these interactions through a combination of in vitro and in silico modeling and data analysis resulting in reverse engineering of the process and then designing an optimal composition in order to yield the most efficacious multi-component formulation (Tam and Tuszynski 2008).

Precision and accuracy of in vitro parameter determinations are essential for the construction of an adequate pharmacokinetic/pharmacodynamic model. Besides absorption, the use of botanical extracts for estimating the rate of hepatic metabolism and efficacy is seriously flawed. A conventional extract may contain substances that are not absorbable but have the capability of interacting with biological processes. This will be a huge source of error in estimating in vivo conditions.

After oral ingestion, an herbal substance or extract has to survive the harsh environment of the gastrointestinal tract. The content may be degraded by the acidic environment of the stomach, by enzymes in the stomach and the intestine and by bacteria in the colon. The active components may be degraded and inactive components may turn into active moieties. The inactive and active components could interact with each other during absorption. Paracelcian (Landes, G. et al. 2000) has used this physiological concept to digest extracts and collect absorbable fractions using Caco-2 as an intestinal mimic. Instead of measuring the actual chemical composition of these fractions, the quality of an extract was measured using a biological assay. The thrust of the standardization process is dependent on the activity of the digested extracts. The absorption mimic was used to confirm that the absorbable fractions are still active. There were no descriptions on the chemical profile of the extract, digested extract or the absorbable fraction. From a pharmaceutical point of view, it is unacceptable because it is impossible to ensure chemical consistency. In this invention, the same physiological approach is used. The difference is that the chemical identity of the bioactives is quantified and the magnitude of interaction with knowns and unknowns are also measured. Chemical identification was acknowledged in Paracelcian's invention as highly complicated, hence, no attempts at identification were made.

Similar to conventional extraction procedures, Pharmcologix (Jia and Hong 2003) was developed as a high throughput system using a two-step extraction process. The solvents used have properties ranging from polar to nonpolar. The technology was aimed at identifying a lead compound and there was no intention to identify a profile of leads, which could interact with each other. Therefore, it has no relevance to the present invention in that the multiple-component approach is the theme of this invention. Furthermore, this invention is focused on developing absorbable components whereas absorption is not a focus of the Pharmacologix technology (Jia and Hong 2003).

### SUMMARY OF THE INVENTION

This invention is about identifying absorbable components in a complex mixture by following the physiological processes of oral absorption. The preparation of extracts containing absorbables and/or precursors of absorbables is achieved by removing the unabsorbables using size exclusion or nano-filtration.

By following the physiological processes of digestion and metabolism, the change in chemical profile of a preparation is revealed. This is achieved by treating polar, semi-polar and non-polar extracts of an herbal products with simulated gastric and intestinal fluids, and colonic bacteria. The absorbable components and/or their precursors are identified using cell monolayers such as that of Caco-2 and MDCK cells. The unabsorbables are separated using molecular fractionation. Extracts designed using this knowledge base contain absorbable components which can either be commercialized or to be used for pharmacokinetic and pharmacological studies.

### DETAILED DESCRIPTION OF THE FIGURES

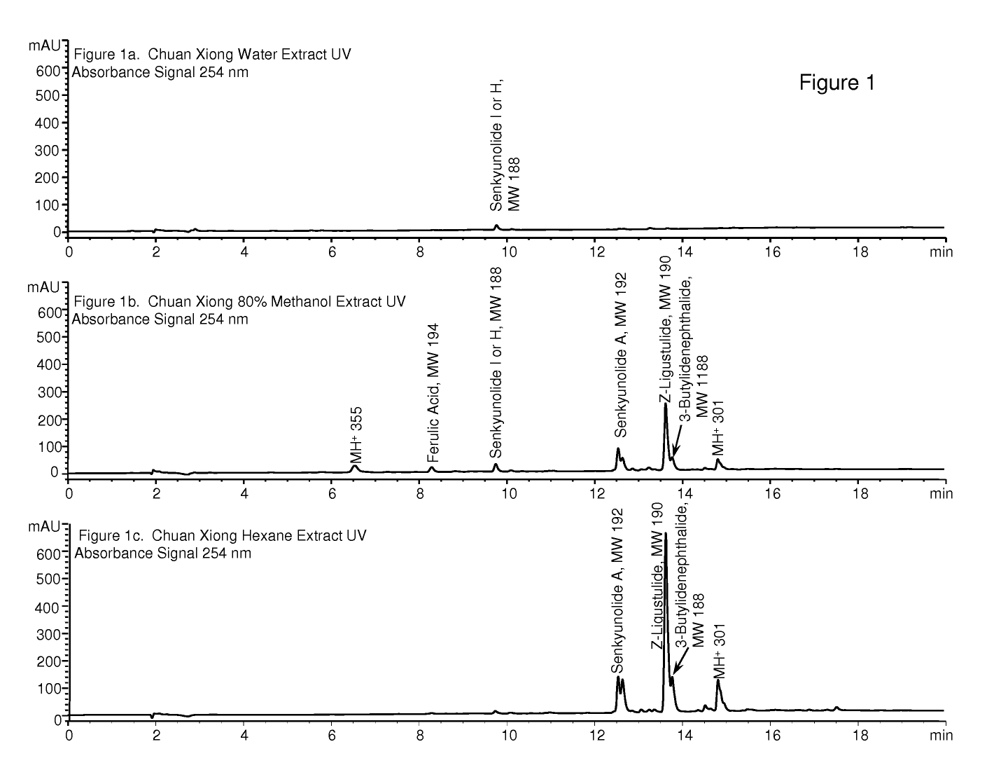

FIG. 1 shows HPLC/UV chromatograms of Rhizoma Chuanxiong extract. a. water extract; b. 80% methanol extract and c. hexane extract. All samples are 1 mg/mL dry extract in the appropriate solvent wherein 20 μL has been injected onto the column. Chromatograms are normalized to 700 mAU.

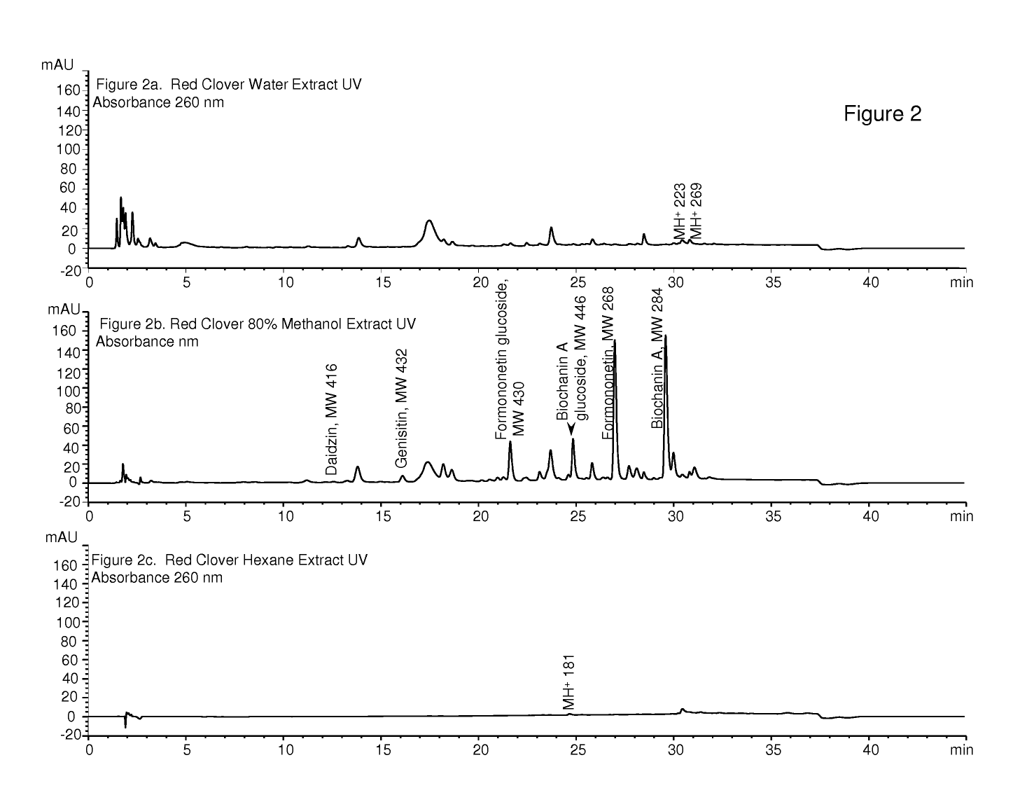

FIG. 2 shows HPLC/UV chromatograms of Red clover extract. a. water extract; b. 80% methanol extract and c. hexane extract. All samples are 1 mg/mL dry extract in the appropriate solvent wherein 20 μL has been injected onto the column. Chromatograms are normalized to 170 mAU.

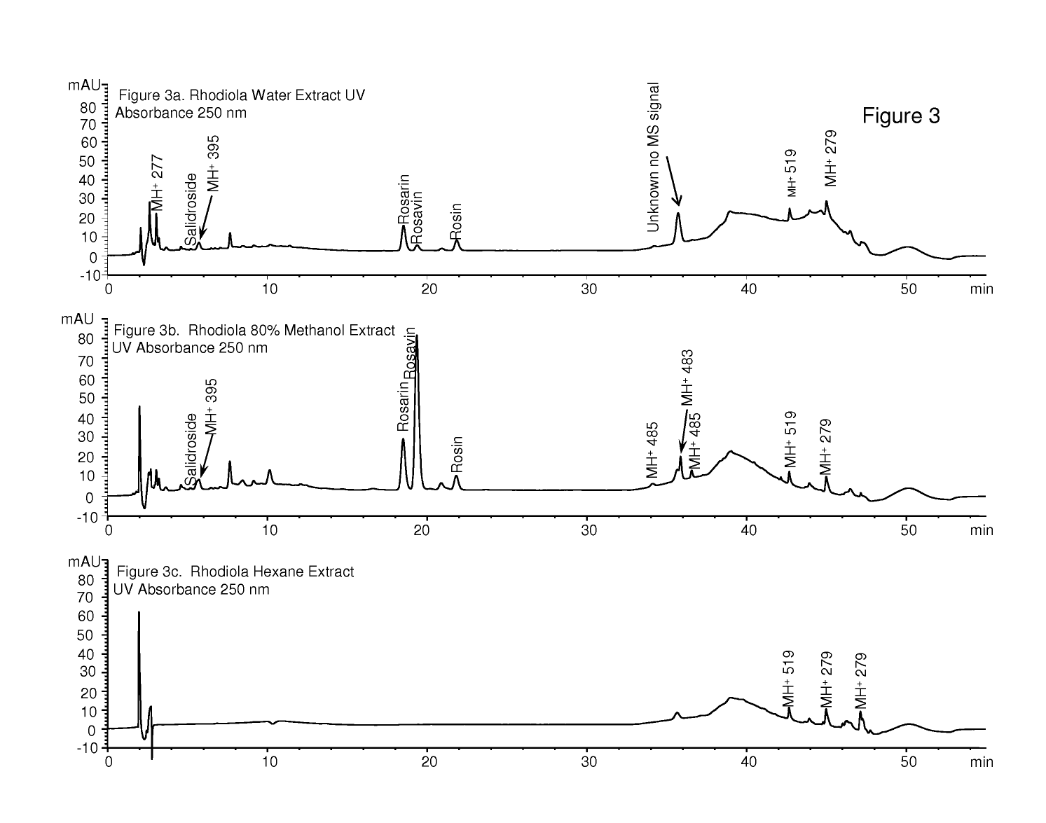

FIG. 3 shows HPLC/UV chromatograms of Rhodiola rosea extract. a. water extract; b. 80% methanol extract and c. hexane extract. All samples are 1 mg/mL dry extract in the appropriate solvent wherein 20 μL has been injected onto the column. Chromatograms are normalized to 90 mAU.

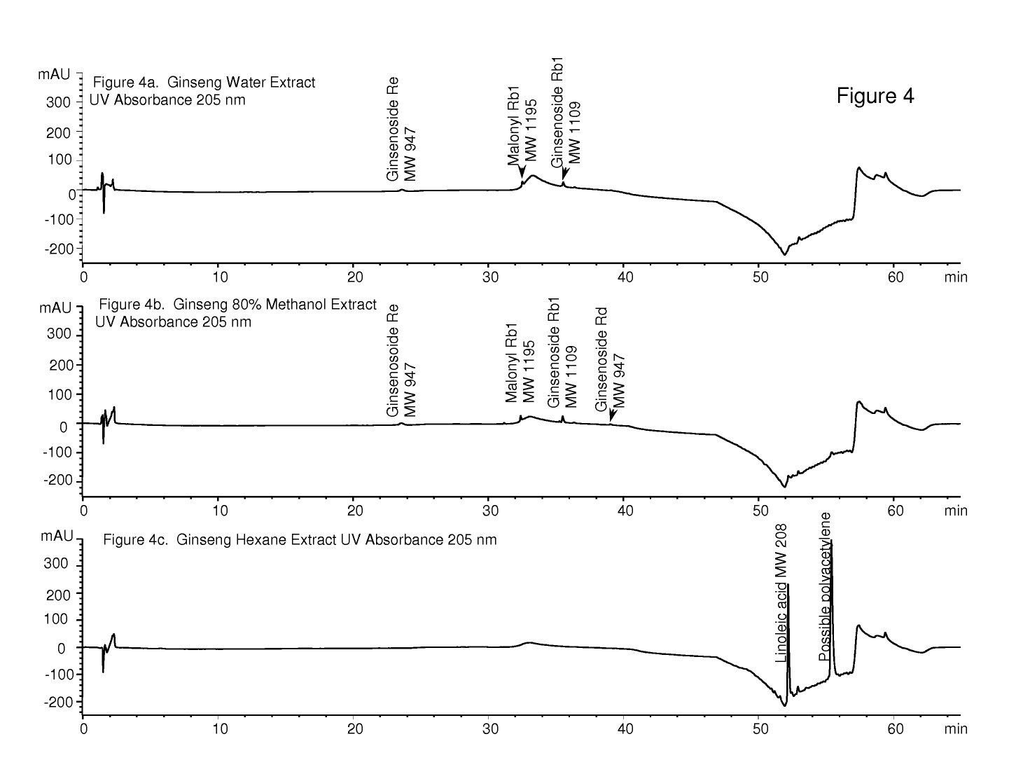

FIG. 4 shows HPLC/UV chromatograms of Panax ginseng extract. a. water extract; b. 80% methanol extract and c. hexane extract. All samples are 1 mg/mL dry extract in the appropriate solvent wherein 20 μL has been injected onto the column. Chromatograms are normalized to 400 mAU.

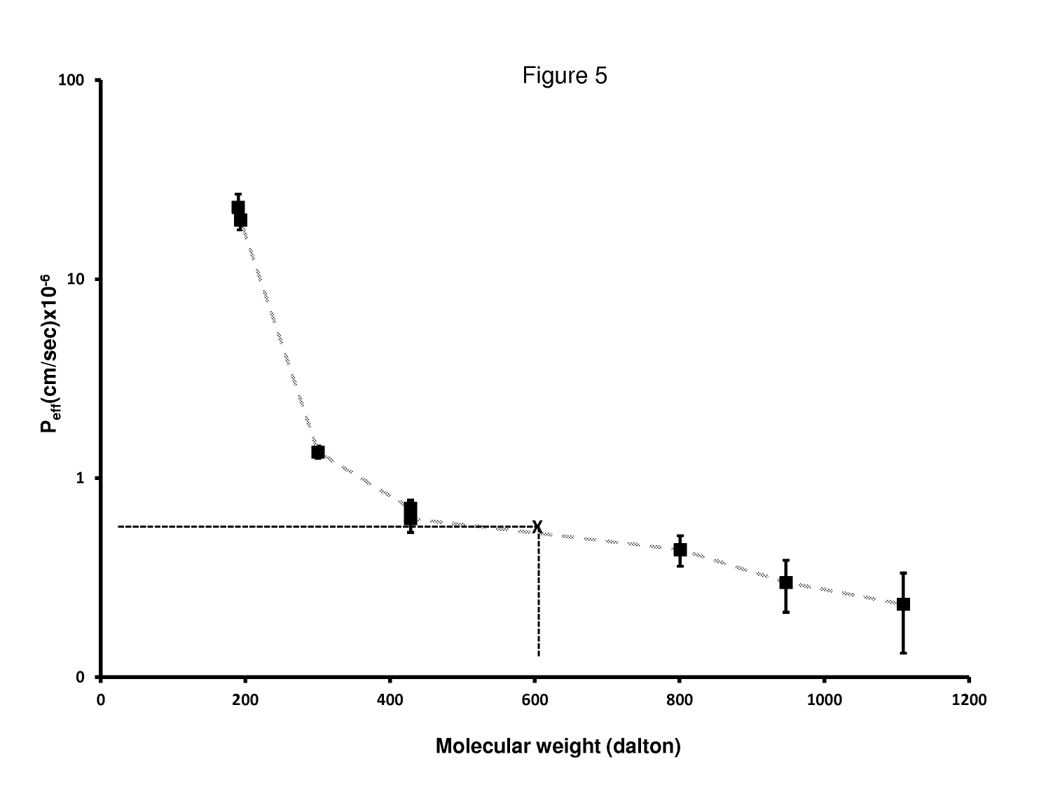

FIG. 5 shows the permeability of 7 purified chemicals on a CaCo-2 cell system demonstrating the relationship between permeability and the molecular weight of a compound. The compounds are Salidroside (M.W. 300.3), rosarin (M.W. 428.4), rosarin (M.W. 428.4), ginsenoside Rg1 (M.W. 801.0), ginsenoside Rb1 (M.W. 1109.3), ginsenoside Rd (M.W. 947.1), senkyunolide A (M.W. 193.2), and z-ligustilide (M.W. 190.2). The CaCo-2 cell system was set up according to Yee (Yee, 1997 #1486). The permeability follows a general downward trend: the larger the molecular weight, the less permeable the compound is. The “X” marks the cut off molecular weight for permeability.

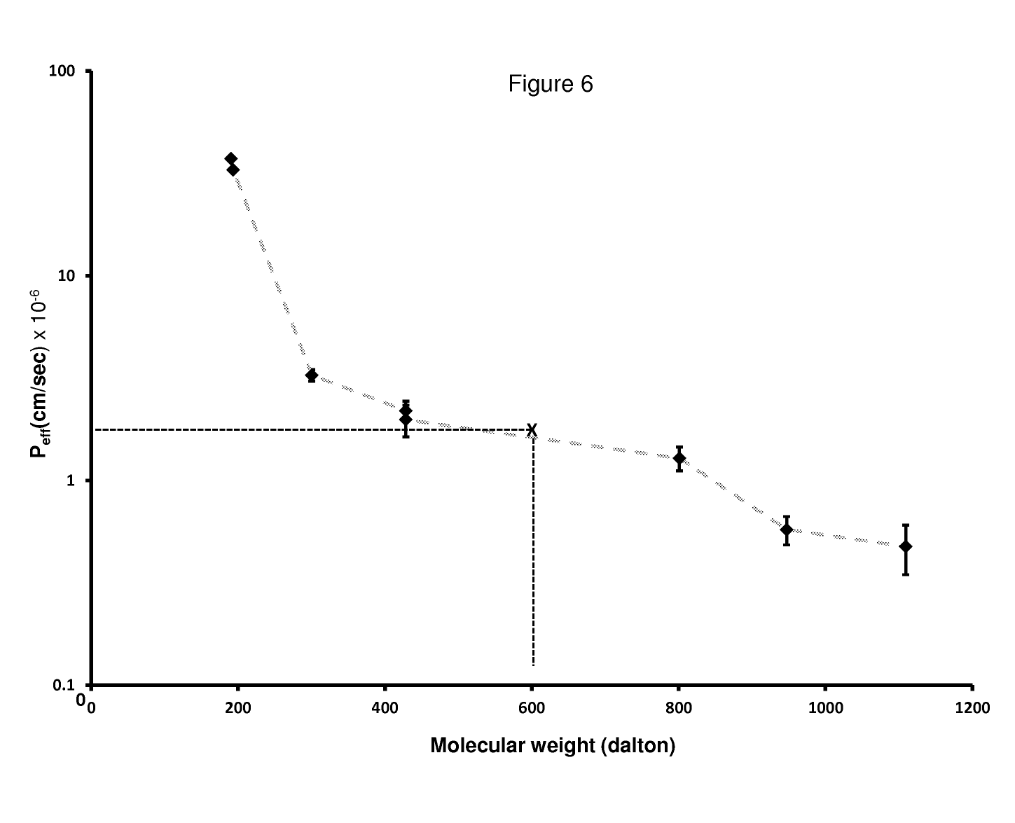

FIG. 6 shows the permeability of 8 purified chemicals on an MDCK cell system demonstrating the relationship between permeability and molecular weight of a compound. The compounds are Salidroside (M.W. 300.3), rosarin (M.W. 428.4), rosavin (M.W. 428.4), ginsenoside Rg1 (M.W. 801.0), ginsenoside Rb1 (M.W. 1109.3), ginsenoside Rd (M.W. 947.1), senkyunolide A (M.W. 193.2), and z-ligustilide (M.W. 190.2). Permeability of these compounds was measured using MDCK cells. The MDCK cell system was set up according to Lin et al. (Lin, Tam et al. 2003). The permeability follows a general downward trend: the larger the molecular weight, the less permeable the compound is. The “X” marks the cut off molecular weight for permeability.

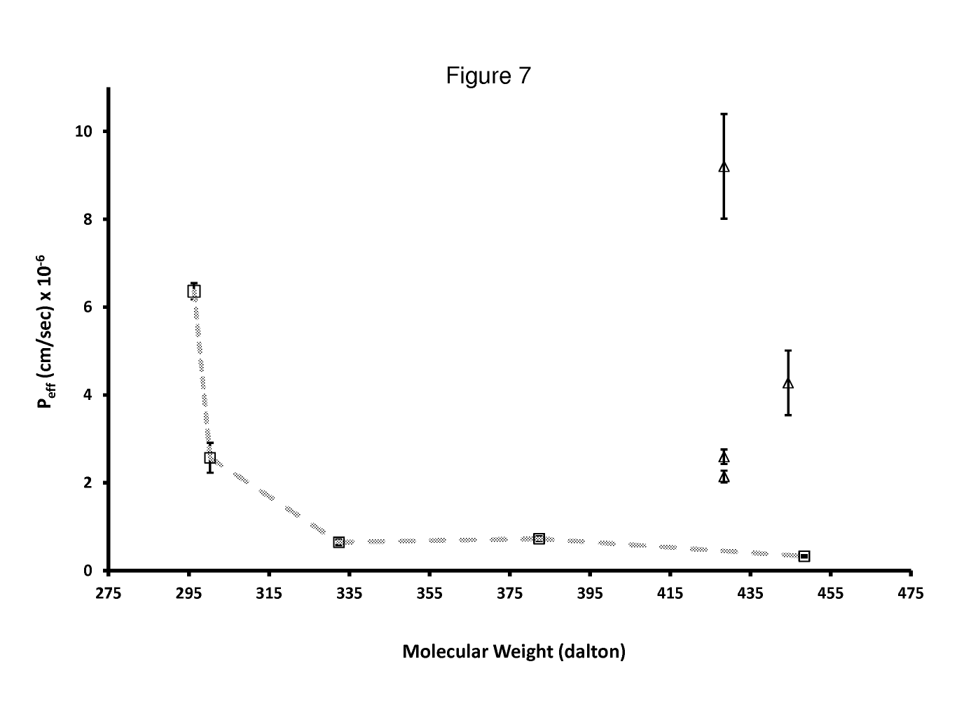

FIG. 7 shows the permeability of Rhodiola components in an 80% methanol extract through MDCK cells. Permeability of five components, rosin (M.W.: 296.3), salidroside (M.W.: 300.3), rosiridin (M.W.: 332.4), UnRT32 (M.W.:382.3), and rosavin related compound (M.W.: 448.4), decreases with an increasing molecular weight (squares) and four components, rosarin (M.W.: 428.4), rosavin (M.W.: 428.4), rosavin isomer (M.W.: 428.4), hydroxyrosavin (M.W.: 444.4), deviated significantly from the trend (triangles).

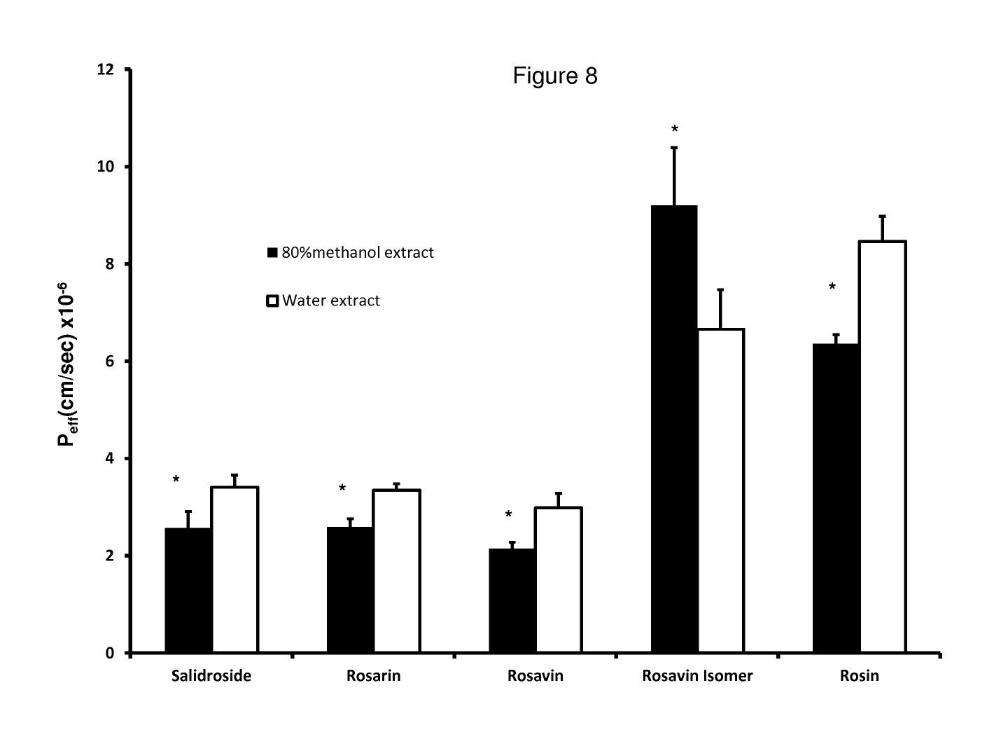

FIG. 8 shows a comparison of permeability of chemicals between 80% methanol and water extracts of Rhodiola. Permeability studies were conducted with an MDCK cell system. Black bars represent chemicals from an 80% Rhodiola methanol extract and white bars represent chemicals from a Rhodiola water extract. The presence of an asterisk (*) indicates that the permeability of the extracts was statistically significant (p<0.05).

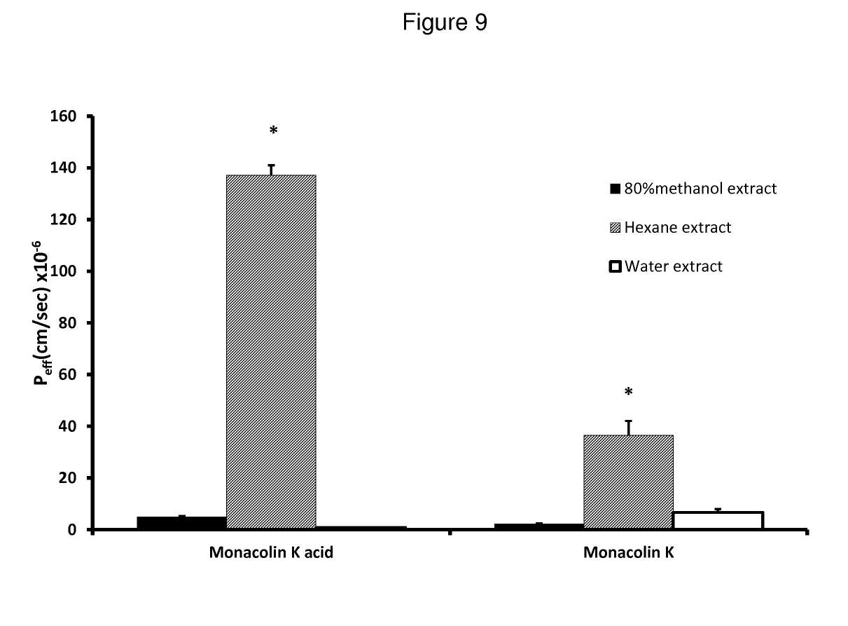

FIG. 9 shows a comparison of the permeability of compounds from 3 different extracts of red yeast rice, specifically, a comparison of the permeability of monacolin K acid and monacolin K extracts from water, 80% methanol and hexane extract. The study was performed using an MDCK cell system. Black bars represent compounds obtained from an 80% methanol extract, grey bars from a hexane extract, and white bars from a water extract. An asterisk (*) indicates that the difference in permeability of the hexane extract was statistically significant (p<0.05) from the rest.

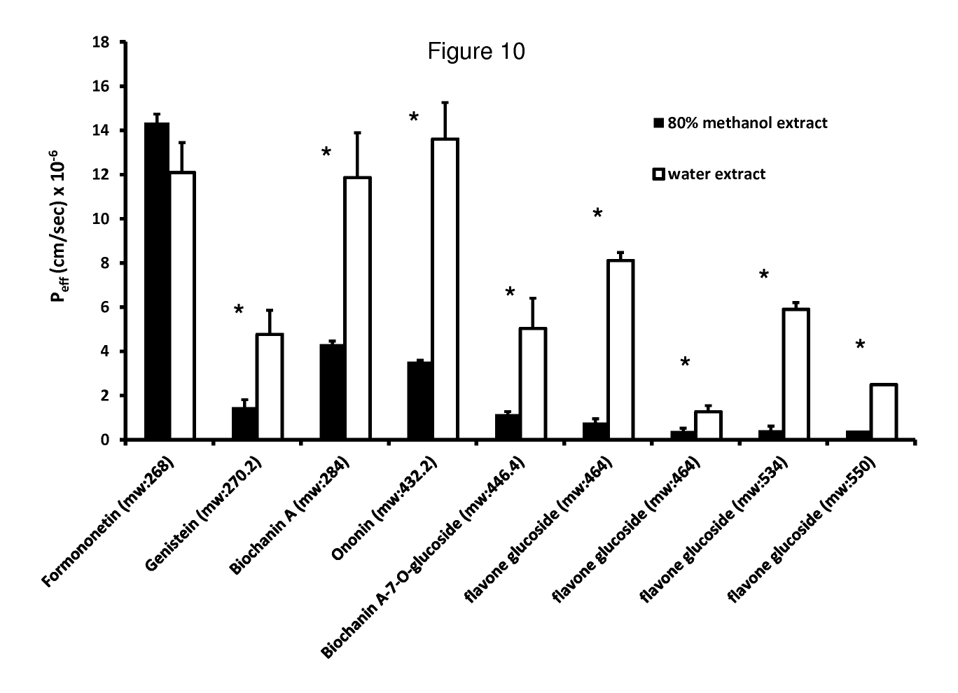

FIG. 10 shows a comparison of permeability between 80% methanol extract and water extract of Red clover. Solid bars represent chemicals from 80% methanol extract and open bars represent chemicals from water extract. An asterisk (*) indicates that the differences in permeability of the extracts were statistically significant (p<0.05).

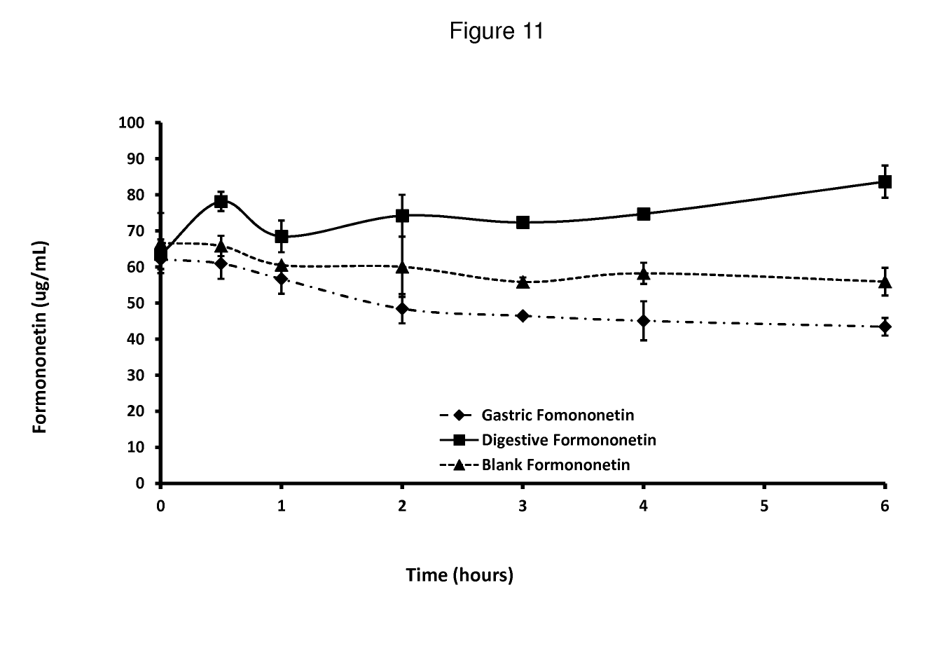

FIG. 11 shows the stability of formononetin in Red Clover 80% methanol extract. Mixture was incubated with Artificial Gastric and Digestive Juices.

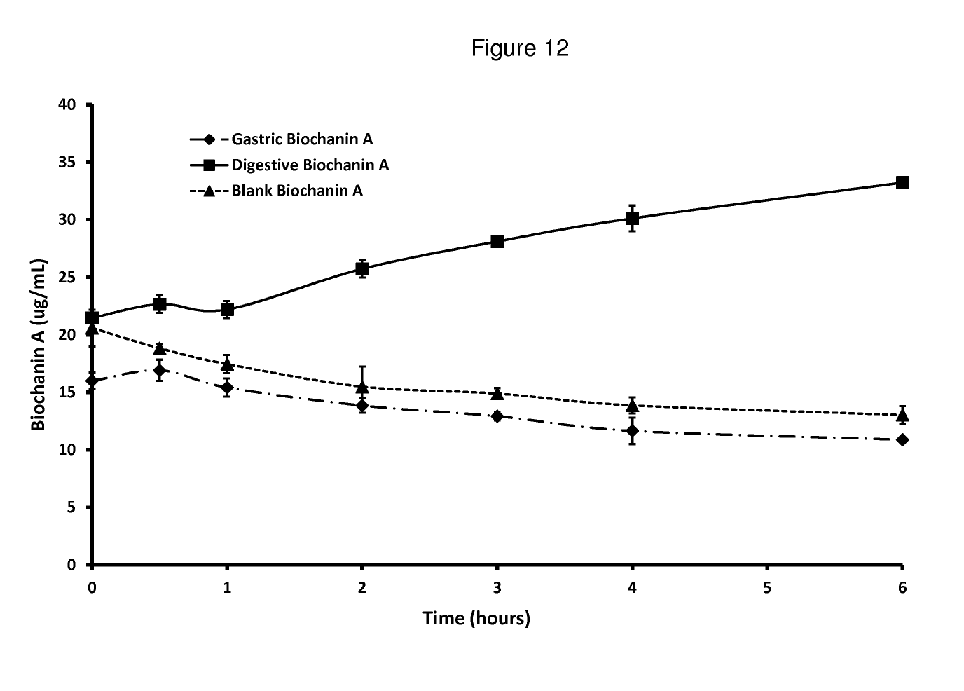

FIG. 12 shows the stability of Biochanin A in Red Clover 80% methanol extract. Mixture was incubated with Artificial Gastric and Digestive Juices.

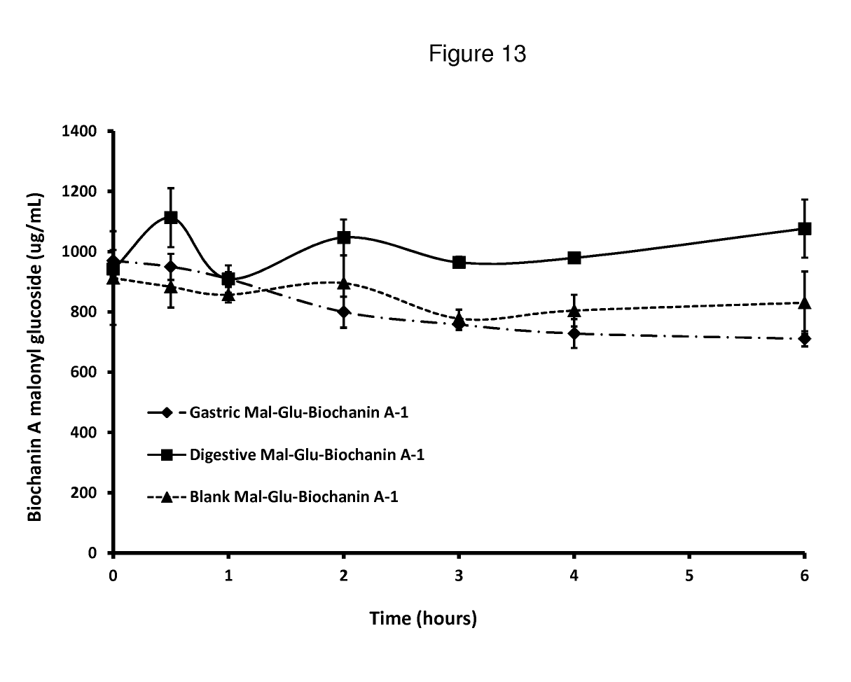

FIG. 13 shows the stability of Biochanin A malonyl glucoside in Red Clover 80% methanol extract. Mixture was incubated with Artificial Gastric and Digestive Juices.

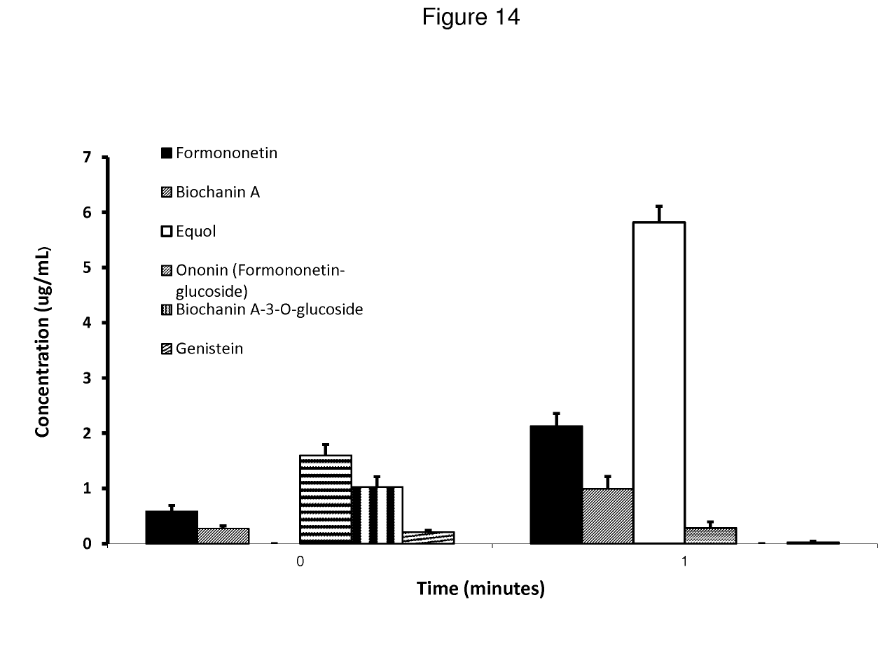

FIG. 14 shows the rapid metabolism of Red clover components in 80% methanol extract by intestinal bacteria.

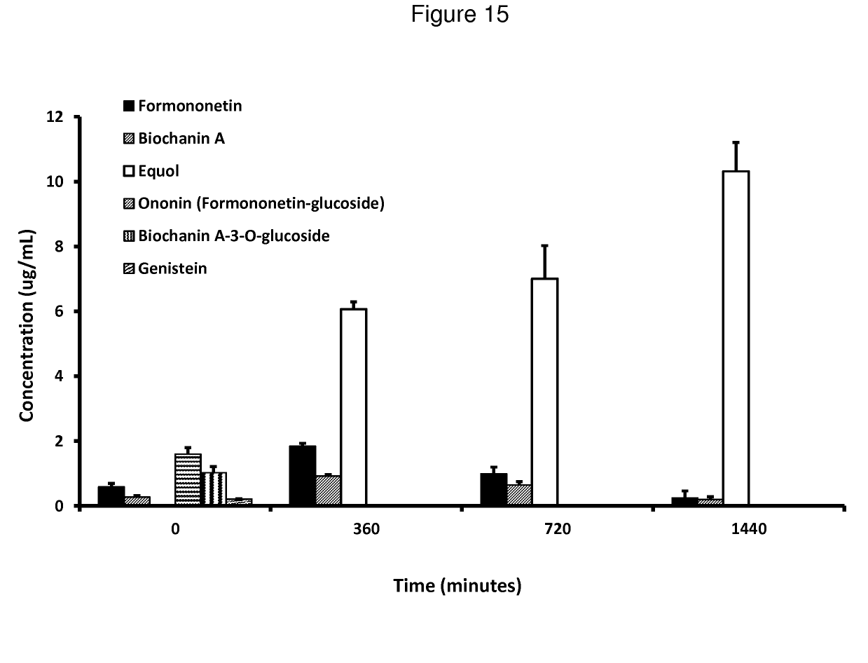

FIG. 15 shows the metabolism of Red clover components in 80% methanol extract by intestinal bacteria (up to 24 hours).

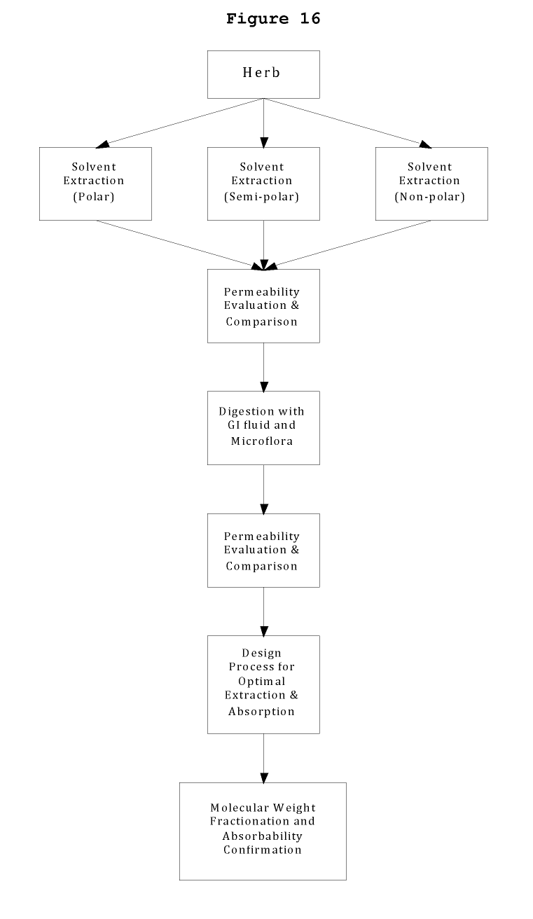

FIG. 16 is a flow chart that shows the optimization process of an herb in terms of extraction efficiency and absorbability.

### DETAILED DESCRIPTION OF THE INVENTION

The most physiological way to prepare an absorbable mimic is to ‘filter’ the digested fraction using a biological membrane such as that of Caco-2 or MDCK cells. However, the yield using this method is miniscule. In this invention, a process for preparing quantitative absorbables is described.

Botanical substances are complex mixtures of compounds with chemical characteristics ranging from polar to non-polar and their molecular weight can span a wide range. In general, small nonpolar compounds are the most absorbable, whereas large polar compounds are unlikely to be absorbed after oral ingestion. Some of these compounds may be digested in the gastrointestinal (GI) tract by acid, enzymes or microflora. The resultant metabolites may have a smaller molecular weight with higher lipophilicity. A good example is the conversion of a glucoside to its respective aglycone. Following the physiological process, a strategy is devised to prepare an absorbable fraction from a complex mixture, which could be an herb, botanical, marine biologicals or animal products (FIG. 16).

An herbal substance is extracted using three types of solvents with properties ranging from polar to nonpolar. The strategy is to segregate the polar from non-polar components while at the same time evaluating the extraction efficiency of each solvent system. The permeability/absorption of these extracts is estimated using a cell system such as MDCK or Caco-2. The permeability of the same components between extracts is compared. This procedure reveals potential interactions during the absorption process. The same procedures are repeated after the extracts are digested using simulated gastric and intestinal fluids, and intestinal bacteria. The intent is to identify potential metabolites, which may be absorbable and/or could interfere with the absorption process. Results obtained from this series of tests are used to design an optimal extraction process, which may involve a combination of extraction steps. At the end, molecular weight fractionation is used to remove substances that are not absorbable and the final product is examined for permeability. The components in this final product are either all absorbables or contain precursors of absorbables.

In one embodiment, the present invention provides a process involving a basic method of designing an optimal extraction method for complex mixtures, wherein the mixture is extracted with polar, semi-polar or non-polar solvents, and the permeability of these extracts is tested, the permeability of substances determined to be permeable from all extracts is further tested and compared, and a solvent system or program is created which extracts all permeable substances from such mixtures in an optimal fashion. The complex mixtures can be, but are not necessarily limited to herbal products, animal products, botanicals, marine biologicals, or synthetic mixtures.

In one embodiment, the permeability of the aforementioned extracts is tested through the use of an in vitro cell monolayer assay. The in vitro cell monolayer assay may utilize cells which are capable of forming a tight junction, e.g. Caco-2, MDCK, or HT29 cells etc.

In one embodiment, the polar solvent or solvents used in the processes can be water, organic acids, organic bases, ketones, aldehydes, sugars, or salts, but is not necessarily limited to the recited solvents.

In one embodiment, the semi-polar solvent may be a hydroalcoholic solution. This hydroalcoholic solution may be, but does not necessarily have to be methanol, ethanol, propranol, or butanol. The hydroalcoholic solution may be a solution wherein the alcohol in the mixture ranges from 10 to 90% of the total solution.

In one embodiment, the non-polar solvent in the process is a water immiscible solvent. Examples of such water immiscible solvent include, but are not limited to, hexane, C5 to C12 alkane, ether, chloroform, ethyl acetate, and dichloromethane.

In one embodiment, the process may further involve digesting extracts with simulated gastric juice, simulated intestinal juice, or intestinal bacteria before permeability is tested. In one embodiment, the simulated gastric juice comprises hydrochloric acid or gastric enzymes. In one embodiment, the pH values of the simulated gasric juices ranges from 1 to 3, whereas the simulated intestinal juice comprises an appropriate buffer with a pH value ranging from 5 to 8. In another embodiment, the simulated intestinal juice includes intestinal or pancreatic enzymes.

In one embodiment, the temperatures under which extraction take place in the recited process range from 20 to 100° C.

In one embodiment, the recited process can involve the removal of impermeable components through molecular weight fractionation. Standard methods of molecular weight fractionation include, but are not limited to, size exclusion chromatography and nano-filtration.

In one embodiment, the processes can be utilized with extracts extracted from Red Clover leaves. In one embodiment, the Red Clover leaf extracts are extracted with water and a mixture of ethanol and water in sequence. For example, the extraction of Red Clover takes place with water for 1 to 6 hours and with an ethanol water mixture for 1 to 6 hours.

In another embodiment, bioactives of Red Clover leaves may be extracted at temperatures ranging from 25 to 80° C.

In another embodiment, the present invention provides a composition prepared according to the optimal extraction processes devised from the method described herein. Such compositions may further be created in which molecules above 500 Daltons have been excluded from the composition by molecular fractionation.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, unrecited elements or method steps.

### EXAMPLE 1

The objective is to provide extracts of biological materials containing soluble compounds of different polarities. Three or more extracts are to be created and they include but are not limited to:

1. An extract containing highly polar (water soluble) molecules. In this case the fraction is created from a biological material extracted with water and will contain water soluble molecules such as but not limited to organic acids, organic bases, alcohols, ketones, aldehydes, sugars and salts.

2. An extract containing moderately polar materials. In this case the fraction is created from a biological material extracted with a hydroalcholic solution and will contain molecules easily soluble in water/alcohol mixtures. Such molecules may include but are not limited to flavones, statins, saponins, polyphenols and any other biological compounds that will dissolve in hydroalcoholic solvents. The hydroalcoholic solution is preferably 80% methanol but other alcohols (ethanol, propanol, butanol, etc.) and solvents may be used and the proportion of solvent can range from 10% to 100%.

3. An extract containing nonpolar compounds. In this case the fraction is created from a biological material extracted with a nonpolar solvent and will contain molecules easily soluble under nonpolar conditions but not soluble under polar conditions. Such molecules may include but are not limited to fats, oils, sterols and fatty acids. The nonpolar solvent is preferably hexane but can be any C₅ to C₁₂ alkane, ether chloroform, ethyl acetate, dichloromethane or any other water immiscible solvent. Supercritical CO₂ extraction may also be employed.

To extract the sample a weighed amount of the material is crushed, ground or homogenized and mixed with a known volume of the desired solvent. The preferred ratio is 1 to 10 sample weight to solvent volume but can range from 1 to 1 up to 1 to 50 weight to volume. Depending on the desired outcome and the solvent conditions the extraction duration and temperature can be varied. Temperature can vary from solvent freezing point to boiling point and above (reflux extraction for example) depending on containment and pressurization.

The extract is then clarified by filtration, centrifugation or other means and stored as a liquid extract or the solvent is removed to concentrate or dry the material. The material is stored for further scientific evaluation (chemical identity, pharmacological action, toxicity, etc.).

Chromatographic profiles of such extracts are provided in the following figures (FIGS. 1-4). These profiles are based on reverse phase separation of the components and the most polar compounds elute at earlier time points than moderately polar compounds. Nonpolar components elute at the longest retention time.

FIG. 1 illustrates the chemical profiles obtained from water extraction (FIG. 1a), 80% methanol extraction (FIG. 1b) and hexane (FIG. 1c) extraction of Chuanxiong (Ligusticum wallichi) rhizome. Water extract (FIG. 1a) of Chuanxiong rhizome contains little in the way of chromatographically observable compounds. The water extract contains little of the characteristic phthalides and is mostly comprised of water soluble sugars, oligosaccharides and organic acids that are not seen by ultraviolet absorbance. The 80% methanol fraction (FIG. 1b) contains numerous moderately polar compounds characteristic of Chuanxiong including ferulic acid and various senkyunolides. Moderate amounts of z-ligustilide and 3-butylidenephthalide are also seen. The hexane extract (FIG. 1c) excludes ferulic acid and some of the senkyunolides while enhancing the content of z-ligustilide and 3-butylidenephthalide and other late retaining materials.

FIG. 2 illustrates the chemical profiles obtained from water extraction (FIG. 2a), 80% methanol extraction (FIG. 2b) and hexane (FIG. 2c) extraction of Red clover (Trifolium pratense) aerial parts. Water extract (FIG. 2a) of red clover contains little in the way of chromatographically observable compounds. The water extract contains little of the characteristic isoflavones and is mostly comprised of water soluble sugars, oligosaccharides and organic acids that are observed close to the solvent front (retention time between 1 and minutes). The 80% methanol fraction (FIG. 2b) contains numerous moderately polar compounds characteristic of red clover including isoflavone glucosides and isoflavone aglycones. The hexane extract (FIG. 2c) excludes the isoflavones. Aerial part extracts of clover contain little if any nonpolar compounds such as phytosterols, lipids and oils and this is borne out by the fact that little is demonstrated in the chromatographic profile of the hexane extract.

FIG. 3 illustrates the chemical profiles obtained from water extraction (FIG. 3a), 80% methanol extraction (FIG. 3b) and hexane (FIG. 3c) extraction of Rhodiola (Rhodiola rosea) roots and crown. Water extract (FIG. 3a) of Rhodiola contains moderate amounts in the way of chromatographically observable compounds including salidroside, gallic acid and rosavins. The 80% methanol fraction (FIG. 3b) contains numerous moderately polar compounds characteristic of Rhodiola including rosavins as well as cinnamic acid, kempferol and cinnamyl alcohol (which are not illustrated). The hexane extract (FIG. 3c) excludes the rosavins and includes at least 3 unidentified nonpolar compounds noted by characteristic molecular weights.

FIG. 4 illustrates the chemical profiles obtained from water extraction (FIG. 4a), 80% methanol extraction (FIG. 4b) and hexane (FIG. 4c) extraction of Ginseng (Panax ginseng) root. Water extract (FIG. 4a) of ginseng contains little in the way of chromatographically observable compounds except minor amounts of ginseng saponins which are soluble in both water and hydroalcoholic solvents. The 80% methanol fraction (FIG. 4b) contains numerous moderately polar compounds characteristic of ginseng including ginsenosides. The hexane extract (FIG. 4c) excludes the ginsenosides and includes at least 2 nonpolar compounds identified as linoleic acid and a ginseng polyacetylene.

### EXAMPLE 2

The objective of this example is to examine the relationship between absorption and the molecular weight of a molecule.

In vitro cell monolayer systems have long been used in the pharmaceutical industry to estimate or predict the absorbability of leads. It is understood that there are at least three pathways through which chemicals traverse through the gut epithelium, i.e., transmembrane, paracellular, and active transporters. The in vitro cell monolayer systems are used to mimic the gut epithelium and estimate/predict the transmembrane and paracellular absorption with reasonable success. Chemicals passing through the gut epithelium through the transmembrane route are non-polar and the permeability is not molecular weight dependent. Chemicals that are absorbed through the paracellular route are more polar and the permeability is size-dependent (the larger the molecule the lower the permeability).

To demonstrate the relationship between permeability and molecular weight, the permeability of pure compounds from plant sources were measured using CaCo-2 and MDCK cell monolayers.

Both CaCo-2 and MDCK cells were purchased form American Type Culture Collection (ATCC) and cultured with Dulbecco Modified Eagle's Media (DMEM) supplemented with 10% fetal bovine serum, 1% non-essential amino acids, and 10 mM HEPES. All tissue culture media and supplements were purchased from Sigma-Aldrich. Following Yee's method (Yee 1997), CaCo-2 cells were seeded into transwell culture dishes and cultured for 21 days. Lin et al's. (2003) method was used to seed and culture MDCK cells; the culturing period was three days.

Salidroside (M.W. 300.3), rosarin (M.W. 428.4), rosarin (M.W. 428.4), ginsenoside Rg1 (M.W. 801.0), ginsenoside Rb1 (M.W. 1109.3), ginsenoside Rd (M.W. 947.1), senkyunolide A (M.W. 193.2), and z-ligustilide (M.W. 190.2) were purchased from Chromadex Inc. These compounds were first prepared in appropriate solvents as stock solutions and diluted in Hanks buffered saline supplemented with 20 mM HEPES to a final concentration of 5 μM each. The duration for the Caco-2 and MDCK cell studies were 120 minutes and 60 minutes, respectively. The samples were mounted on a shaker running at 50-70 rpm and the temperature was kept at 37° C.

Samples were taken from the apical side (donor) at 0 and 120 minutes and at 120 minutes from basal side (receiver) for CaCo-2 cells; and at 0 minute and 60 minutes from apical side (donor) and at 60 minutes from basal side (receiver) for MDCK cells after incubation. Samples were diluted with a 1:1 (v/v) mixture of methanol and acetonitrile and analyzed using a LC/MS system. Permeability coefficient number was calculated as followed: P_{eff} (cm/sec)=(Vᵣ/(A*Cₛ))*(C_{f}/t), where Vᵣ is the receiver side volume (basal; ml), A is the area of monolayer (cm²), Cₛ is the starting concentration at the donor side (μM), C_{f} is the concentration at the receiver side at time t (μM), and t is the incubation time (seconds).

Permeability results from CaCo-2 cells and MDCK cells show the similar trend: the higher the molecular weight the lower the permeability (FIGS. 5 and 6). Based on the systems established in our laboratory and reported literature, compounds with permeability coefficient, P_{eff} (cm/sec) less than 5E-7 cm/sec (for CaCo-2 system) and 2E-6 cm/sec (for MDCK) are considered low to impermeable. The molecular weight cut-off is approximately 600 Dalton. It should be noted that these numbers were established using pure compounds. Actual permeability of a compound may vary in a complex mixture. The reason is that the other components in the mixture may interfere with the absorption process.

### EXAMPLE 3

The objective of this example is to show that the permeability of a component in complex mixture similar to that of an herbal extract could vary, depending on how the component is extracted.

Two natural products: Rhodiola rosea and Red yeast rice were individually extracted using the three solvents described in Example 1. Each extract was dissolved in Hanks' balanced salt buffer supplemented with 20 mM HEPES at 1 mg/ml. Permeability studies were carried out using MDCK cells and samples were taken from the apical side at 0 minute and 60 minutes and from the basal side at 60 minutes after incubation. Samples were diluted with a 1:1 mixture of methanol and acetonitrile prior to analysis using a LC/MS system. Permeability coefficient number was calculated as follows: P_{eff} (cm/sec)=(Vᵣ/(A*Cₛ))*(C_{f}/t), where Vᵣ is the receiver side volume (basal; ml), A is the area of monolayer (cm²), Cₛ is the starting concentration at the donor side (μM), C_{f} is the concentration at the receiver side at time t (μM), and t is the incubation time (seconds).

In general, the permeability values of nine Rhodiola rosea components in the methanolic extract were found to decrease with increasing molecular weight, except rosarin, rosavin, rosavin isomer and hydroxyrosavin (FIG. 7). These four compounds have a molecular weight higher than 400 Daltons. It is likely that these compounds are absorbed via active transport. The permeability of these compounds was found to be significantly different when they were extracted with 80% methanol as opposed to that of water (FIG. 8), suggesting the absorption of these compounds was interfered by co-existing components. These components could either be known or unknown.

In the case of Red yeast rice, it was found that the permeability of monacolin K acid and monacolin K was the highest in the hexane extract (FIG. 9).

The results of this example clearly show that standardization of a bioactive in an herbal preparation does not guarantee consistency in terms of efficacy. At the very least, the bioavailability of a bioactive can be altered by the presence of other substances, which could be dependent on the extraction method used.

### EXAMPLE 4

The objective of this example is to prepare an absorbable fraction of Red clover using the method described in this invention.

### Permeability of Major Components

Approximately one gram of dried leaves of Red clover was extracted with either 10 volumes of water, 80% methanol or hexane at 60° C. for 3 hours. The extracts were using a rotor-evaporator. The dry extracts were dissolved in appropriate solvents to provide 1 mg/ml samples. Twenty IL was injected onto an HPLC equipped with a UV and an electrospray mass spectrometric detector. The methanolic extract provided the highest yield of known isoflavones, which are the major bioactives (FIG. 2). Hexane extraction yielded the poorest recovery.

The extracts of Red clover were individually dissolved in Hanks' balanced salt buffer supplemented with 20 mM HEPES at 1 mg/ml. Permeability studies were carried out using MDCK cells and samples were taken from the apical side at 0 minute and 60 minutes and from the basal side at 60 minutes after incubation. Samples were diluted with a 1:1 mixture of methanol and acetonitrile prior to analysis using a LC/MS system. Permeability coefficient number was calculated as follows: P_{eff} (cm/sec)=(Vᵣ/(A*C,))*(C_{f}/t), where Vᵣ is the receiver side volume (basal; ml), A is the area of monolayer (cm²), Cₛ is the starting concentration at the donor side (μM), C_{f} is the concentration at the receiver side at time t (μM), and t is the incubation time (seconds).

Similar to the observations from Rhodiola and Red yeast rice, there are significant differences in the permeability of major Red clover components (p<0.05), which have been shown to be bioactive (FIG. 10). Components in the water extract tend to enhance eight of the nine measurable components. It should be pointed out that the absorption of glucosides was controversial. There are reports suggesting that these substances are absorbable, whereas the others reported the opposite. This set of results clearly showed that the glucosides present in the methanolic extract are not absorbable. However, in the mix of a water extract, these components become absorbable.

### Stability in Gastrointestinal Fluid and Intestinal Microflora

The GI stability of a methanolic extract of Red clover leaves was evaluated. The decomposition products and/or metabolites formed were also measured.

After oral administration, physiological processes were mimicked by preparing three or more preparations representative of 1) gastric, 2) digestive (pancreatic and/or bile digestion) and 3) colon (anaerobic bacterial) digestion either individually or in in sequence in order to determine what changes if any occur to characteristic chemicals during digestion.

The methanolic extract of Red clover was incubated with simulated gastric juice (Sloley, Lin et al. 2006) at 37° C. for a period of 6 hours. Results show that formononetin and biochanin A, major bioactives in Red clover, were relatively stable under the acidic environment of the stomach (FIGS. 11 and 12). The malonyl glucoside of biochanin A was also stable under the acidic environment of the stomach (FIG. 13).

The methanolic extract of Red clover was incubated with simulated intestinal fluid containing pancreatin. The mixture was incubated at 37° C. for a period of 6 hours. A slight increase in the formononetin level was observed; presumably it was due to the release of formononetin from its precursor glucosides (FIG. 11). The increase in the biochanin A level was more pronounced (FIG. 12); again, it was due to the release of biochanin A from some of its precursors. The malonyl glucoside biochanin A was found to be relatively stable in the simulated intestinal fluid (FIG. 13).

The effect of colonic bacteria on the stability of a methanolic extract of Red clover was evaluated. The procedures reported by Schneider et al. (Schneider, Simmering et al. 2000) was used to prepare a fecal sample collected from a rat. The extract was incubated anaerobically at 37° C. for a period of 24 hours.

The sugar conjugates of formononetin and biochanin A were hydrolyzed very rapidly by the colonic bacteria (FIG. 14). The cleavage of the sugar moiety from these conjugates was reflected by the increase in formononetin and biochanin A levels. Equol, the Phase I metabolite of the metabolite of formononetin, daidzein, also appeared shortly after incubation.

After 6 hours of incubation, the major species left in the incubate was formononetin, biochanin A and equol (FIG. 15).

Taken together, the GI stability studies show that the major components of Red clover are relatively stable in the gastric and intestinal environment. Most of the metabolic conversions occur in the colon. Interestingly, only conjugates of formononetin, biochanin A and equol were found in the plasma after an extract of Red clover was administered to a rat. This set of results suggests that the in vitro studies reflect the in vivo situation quite nicely.

This set of results shows that molecules larger 400 Dalton are unabsorbable when Red clover is extracted with hydroalcoholic solutions. An absorbable fraction can be prepared by fractionating the hydroalcoholic extract using a molecular size cutoff of 400 Dalton.

Varying the concentration of alcohol and temperature could enhance the yield of absorbable components. We found that the highest yield was obtained when Red clover leaves were extracted for three hours with 20% ethanol at 80° C. An absorbable fraction could be prepared by removing molecules higher than 400 Daltons using methodologies such as but not limited to size exclusion and nano filtration. The major components of this extract are formononetin and biochanin A.

The yield of formononetin and biochanin A can be increased by pre-treating Red clover leaves. Leaves stored at −5° C. for a few days will release the aglycones from their respective glucosides completely (Tsao, Papadopoulos et al. 2006).

This set of results has also taught another method of preparing a Red clover extract. The idea is to prepare an extract, which contains components, which promote the absorption of the glucosides. Red clover leaves are extracted a program extraction method. The initial condition of extraction is to use water and the ethanol concentration can be programmed to increase up to 20%. The temperature will be kept at 80° C. and the duration of extraction will be 6 hours. The resultant extract will contain all the glucosides and the aglycones. The components that promote the absorption of the glucosides are also present. This extract will be subjected to molecular fractionation and the cut off is set at 600 Daltons.

A process describing approaches to prepare herbal extracts containing absorbables and/or precursors of absorbables are described. The procedures are based on the physiological events in the gastrointestinal tract. Improvement of component absorption can also be achieved by designing an appropriate extraction condition.

### REFERENCES

Ahlemeyer, B. and J. Krieglstein (2003). “Pharmacological studies supporting the therapeutic use of Ginkgo biloba extract for Alzheimer's disease.” Pharmacopsychiatry 36 Suppl 1: S8-14.
Blumenthal, M. and B. Milot (2004). “Bioassays for testing activity and bioavailability of botanical products.” Journal of the American Botanical Council (63): 48-51.
Brazier, N. C. and M. A. Levine (2003). “Drug-herb interaction among commonly used conventional medicines: a compendium for health care professionals.” Am J Ther 10(3): 163-169.
Chang, T. K., J. Chen, et al. (2006). “Distinct role of bilobalide and ginkgolide A in the modulation of rat CYP2B1 and CYP3A23 gene expression by Ginkgo biloba extract in cultured hepatocytes.” Drug Metab Dispos 34(2): 234-242.
Cheng, Y., Y. Wang, et al. (2006). “A causal relationship discovery-based approach to identifying active components of herbal medicine.” Comput Biol Chem 30(2): 148-154.
Chou, T. C. (2006). “Theoretical basis, experimental design, and computerized simulation of synergism and antagonism in drug combination studies.” Pharmacol Rev 58(3): 621-681.
Hasegawa, H. (2004). “Proof of the mysterious efficacy of ginseng: basic and clinical trials: metabolic activation of ginsenoside: deglycosylation by intestinal bacteria and esterification with fatty acid.” J Pharmacol Sci 95(2): 153-157.
Hollman, P. C., J. M. van Trijp, et al. (1997). “Relative bioavailability of the antioxidant flavonoid quercetin from various foods in man.” FEBS Lett 418(1-2): 152-156.
Homma, M., K. Oka, et al. (1992). “A strategy for discovering biologically active compounds with high probability in traditional Chinese herb remedies: an application of saiboku-to in bronchial asthma.” Anal Biochem 202(1): 179-187.
Hu, Z., X. Yang, et al. (2005). “Herb-drug interactions: a literature review.” Drugs 65(9): 1239-1282.
Jia, Q. and M.-F. Hong (2003). International PCT Publication No. WO/2003/002134. Method for generating, screening and dereplicating natural product librabries for the discovery of therapeutic agents. WIPO: 1-109.
Khwaja, T. A. and E. P. Friedman (2000). U.S. Pat. No. 6,113,907. Pharmaceutical grade St. John's Wort. USPTO Patent Full-Text and Image Database. USPTO.
Khwaja, T. A. and E. P. Friedman (2002). U.S. Pat. No. 6,379,714. Pharmaceutical grade botanical drugs. USPTO Patent Full-Text and Image Database. USPTO.
Komoroski, B. J., R. A. Parise, et al. (2005). “Effect of the St. John's wort constituent hyperforin on docetaxel metabolism by human hepatocyte cultures.” Clin Cancer Res 11(19 Pt 1): 6972-6979.
Komoroski, B. J., S. Zhang, et al. (2004). “Induction and inhibition of cytochromes P450 by the St. John's wort constituent hyperforin in human hepatocyte cultures.” Drug Metab Dispos 32(5): 512-518.
Kwak, W. J., C. K. Han, et al. (2002). “Effects of Ginkgetin from Ginkgo biloba Leaves on cyclooxygenases and in vivo skin inflammation.” Planta Med 68(4): 316-321.
Landes, B., W. G., et al. (2000). International PCT Publication No. WO/2000/003725. Methods for identifying and confirming consistent bio-functionality of natural compositions. WIPO: 1-83.
Lin, Y.-C. J., Y. K. Tam, et al. (2003). International PCT Publication No. WO/2004/018657. Model epithelial cell cultures. WIPO.
Liu, Y. and L. Yang (2006). “Early metabolism evaluation making traditional Chinese medicine effective and safe therapeutics.” J Zhejiang Univ Sci B 7(2): 99-106.
Mathews, J. M., A. S. Etheridge, et al. (2002). “Inhibition of human cytochrome P450 activities by kava extract and kavalactones.” Drug Metab Dispos 30(11): 1153-1157.
Mathews, J. M., A. S. Etheridge, et al. (2005). “Pharmacokinetics and disposition of the kavalactone kawain: interaction with kava extract and kavalactones in vivo and in vitro.” Drug Metab Dispos 33(10): 1555-1563.
Mohutsky, M. A., G. D. Anderson, et al. (2006). “Ginkgo biloba: evaluation of CYP2C9 drug interactions in vitro and in vivo.” Am J Ther 13(1): 24-31.
Newman, D. J., G. M. Cragg, et al. (2003). “Natural products as sources of new drugs over the period 1981-2002.” J Nat Prod 66(7): 1022-1037.
Pan, J. Y. and Y. Y. Cheng (2006). “Identification and analysis of absorbed and metabolic components in rat plasma after oral administration of ‘Shuangdan’ granule by HPLC-DAD-ESI-MS/MS.” J Pharm Biomed Anal 42(5): 565-572.
Pang, P. K. T., J. J. Shan, et al. (2000). Chemical and pharmacological standardization of herbal extracts. USPTO Patent Full-Text and Image Database. USPTO. U.S.A., CV Technologies Inc.
Schneider, H., R. Simmering, et al. (2000). “Degradation of quercetin-3-glucoside in gnotobiotic rats associated with human intestinal bacteria.” J Appl Microbiol 89(6): 1027-1037.
Schulz, H. U., M. Schurer, et al. (2005). “Investigation of pharmacokinetic data of hypericin, pseudohypericin, hyperforin and the flavonoids quercetin and isorhamnetin revealed from single and multiple oral dose studies with a hypericum extract containing tablet in healthy male volunteers.” Arzneimittelforschung 55(10): 561-568.
Sloley, B. D., Y. C. Lin, et al. (2006). “A method for the analysis of ginsenosides, malonyl ginsenosides, and hydrolyzed ginsenosides using high-performance liquid chromatography with ultraviolet and positive mode electrospray ionization mass spectrometric detection.” J AOAC Int 89(1): 16-21.
Tam, Y. K. and K. E. Anderson (2000). U.S. Pat. No. 6,022,733. Simulated biological dissolution and absorption system. USPTO Patent Full-Text and Image Database. USPTO.
Tam, Y. K. and J. A. Tuszynski (2008). International PCT Publication No. WO/2008/120105. Pharmaceutical platform technology for the development of natural products. WIPO.
Tsao, R., Y. Papadopoulos, et al. (2006). “Isoflavone profiles of red clovers and their distribution in different parts harvested at different growing stages.” J. Agric. Food Chem. 54: 5797-5805.
Venkataramanan, R., B. Komoroski, et al. (2006). “In vitro and in vivo assessment of herb drug interactions.” Life Sci 78(18): 2105-2115.
Venkataramanan, R., V. Ramachandran, et al. (2000). “Milk thistle, a herbal supplement, decreases the activity of CYP3A4 and uridine diphosphoglucuronosyl transferase in human hepatocyte cultures.” Drug Metab Dispos 28(11): 1270-1273.
Wang, Y., X. Wang, et al. (2006). “A computational approach to botanical drug design by modeling quantitative composition-activity relationship.” Chem Biol Drug Des 68(3): 166-172.
Williamson, E. M. (2001). “Synergy and other interactions in phytomedicines.” Phytomedicine 8(5): 401-409.
Williamson, E. M. (2005). “Interactions between herbal and conventional medicines.” Expert Opin Drug Saf 4(2): 355-378.
Yan, R., N. L. Ko, et al. (2008). “Pharmacokinetics and metabolism of ligustilide, a major bioactive component in Rhizoma Chuanxiong, in the rat.” Drug Metab Dispos 36(2): 400-408.
Yan, R., G. Lin, et al. (2007). “Low Oral Bioavailability and Pharmacokinetics of Senkyunolide A, a Major Bioactive Component in Rhizoma Chuanxiong, in the Rat.” Ther Drug Monit 29(1): 49-56.
Yee, S. (1997). “In vitro permeability across Caco-2 cells (colonic) can predict in vivo (small intestinal) absorption in man—fact or myth.” Pharm Res 14(6): 763-766.
Yim, J. S., Y. S. Kim, et al. (2004). “Metabolic activities of ginsenoside Rb1, baicalin, glycyrrhizin and geniposide to their bioactive compounds by human intestinal microflora.” Biol Pharm Bull 27(10): 1580-1583.
Zhang, J. L., M. Cui, et al. (2005). “Chemical fingerprint and metabolic fingerprint analysis of Danshen injection by HPLC-UV and HPLC-MS methods.” J Pharm Biomed Anal 36(5): 1029-1035.
Zhou, S., M. Huang, et al. (2005). “Prediction of herb-drug metabolic interactions: a simulation study.” Phytother Res 19(6): 464-471.

What is claimed is:

1. A method of designing an optimal extraction method for complex mixtures, said method comprises the steps of:
a) extracting a mixture with polar, semi-polar or non-polar solvents;
b) testing the permeability of the extracts of step (a);
c) comparing permeability of permeable substances of all extracts; and
d) composing a solvent system or program that extracts all the permeable substances optimally.

2. The method of claim 1, wherein the permeability of the extracts is tested using an in vitro cell monolayer assay.

3. The method of claim 1, wherein the extracts are digested with simulated gastric juice, simulated intestinal juice, or intestinal bacteria before testing of permeability.

4. The method of claim 1, further comprising a step of removing impermeable components by molecular weight fractionation.

5. The method of claim 1, wherein the complex mixtures are selected from the group consisting of herbal products, animal products, botanicals, marine biologicals, and synthetic mixtures.

6. The method of claim 1, wherein the polar solvent is selected from the group consisting of water, organic acids, organic bases, ketones, aldehydes, sugars, and salts.

7. The method of claim 1, wherein the semi-polar solvent comprises hydroalcoholic solution.

8. The method of claim 7, wherein the hydroalcoholic solution is selected from the group consisting of methanol, ethanol, propranol, and butanol.

9. The method of claim 8, wherein the hydroalcoholic solution is a solution wherein the alcohol in the mixture ranges from 10 to 90% of the total solution.

10. The method of claim 1, wherein the non-polar solvent is a water immiscible solvent.

11. The method of claim 10, wherein the water immiscible solvent is selected from the group consisting of hexane, C₅ to C₁₂ alkane, ether, chloroform, ethyl acetate, and dichloromethane.

12. The method of claim 1, wherein the temperatures under which extraction take place range from 20 to 100° C.

13. The method of claim 2, wherein the in vitro cell monolayer comprises Caco-2, MDCK, or HT29 cells which are capable of forming a tight junction.

14. The method of claim 3, wherein the simulated gastric juice comprises hydrochloric acid and gastric enzymes.

15. The method of claim 14, wherein the pH values of the simulated gastric juices range from 1 to 3.

16. The method of claim 3, wherein the simulated intestinal juice comprises an appropriate buffer with a pH value ranging from 5 to 8.

17. The method of claim 16, wherein the simulated intestinal juice includes intestinal or pancreatic enzymes.

18. The method of claim 4, wherein molecular weight fractionation is achieved by size exclusion chromatography or nano-filtration.

19. The method of claim 1, wherein the extracts are extracted from Red Clover leaves.

20. The method of claim 19 comprising extracting Red Clover with water and a mixture of ethanol and water in sequence.

21. The method of claim 19 comprising extraction of Red Clover with water for 1 to 6 hours and with an ethanol water mixture for 1 to 6 hours.

22. The method of claim 19 comprising extracting bioactives of Red Clover leaves at temperatures ranging from 25 to 80° C.

23. A composition prepared according to the optimal extraction method devised by the method recited in claim 1.

24. The composition according to claim 23 wherein molecules above 500 Daltons are excluded from the composition by molecular fractionation.
